(19) United States
(12) Patent Application Publication
(10) Pub. No.: US 2011/0038934 A1
(43) Pub. Date: Feb. 17, 2011

(54) PHARMACEUTICAL COMPOSITION WITH ATORVASTATIN ACTIVE INGREDIENT

(76) Inventors: Alena Prokopova, Praha (CZ); Pavel Sebek, Praha (CZ); Anna Hanovska, Praha (CZ); Michaela Dubovska, Praha (CZ); Vaclav Tomasek, Praha (CZ)

Correspondence Address: Pearl Cohen Zedek Latzer, LLP, 1500 Broadway, 12th Floor, New York, NY, 10036, US

(21) Appl. No.: 12/528,643

(22) PCT Filed: Mar. 3, 2008
(86) PCT No.: PCT/CZ2008/000024

§ 371 Date: Apr. 21, 2010

(30) Foreign Application Priority Data
Mar. 2, 2007 (CZ) PV 2007-169

(51) Int. Cl. A61K 9/28; A61K 31/40; A61K 9/36; A61P 3/06

(52) U.S. Cl. 424/480; 424/474; 514/423

(57) ABSTRACT

A pharmaceutical composition containing the active substance atorvastatin in the form of oblong-shaped tablets with the length of 5 to 22 mm and the width of 2 to 11 mm or round tablets with the diameter of 3 to 16 mm, the core of which is constituted of compressed granulate and contains: i. Atorvastatin and/or at least one physiologically acceptable salt thereof in the quantity of 5 to 10% by weight, related to pure atorvastatin; ii. An organic or inorganic base selected from meglumine or an alkali metal hydroxide or their combination in the quantity of 0.01 to 7% by weight; iii. A pharmaceutically acceptable filler in the quantity of 20 to 90% by weight; iv. A disintegrant in the quantity of 0.5 to 50% by weight; provided with a coat that makes up 1 to 15% of the weight of the core, the selected base being uniformly distributed in the tablet core by means of spraying the same on the solid mixture in the granule production process. The composition can be obtained by a procedure consisting of the following steps: i. Mixing a mixture of atorvastatin, a filler and a disintegrant; ii. Dissolving a base in a mixture of water and a C₁ to C₃ alcohol in the quantity of 10:90 to 90:10 (water/alcohol, by weight); iii. Spraying the dry mixture with the base solution either in a masticating or fluidizing device; iv. Adapting the size of particles of the resulting granulate, preferably by re-sieving to the granule size of 0.1 to 1.5 mm; v. Adding other extragranular components to the granulate that will further improve its ability to be compressed into tablets; vi. Compressing the mixture; and vii. Applying a coat on the compressed tablets.

### TECHNICAL FIELD

The invention relates to a pharmaceutical composition containing a substance with well-known anti-hyperlimidemic effects—atorvastatin. The composition has excellent properties both from the point of view of physical characteristics and as regards the speed of releasing the active substance or product stability.

### BACKGROUND ART

The hemi-calcium salt of (3R,5R) 7-[3-phenyl-4-phenylcarbamoyl-2-(4-fluorophenyl)-5-isopropylpyrrol-1-yl]-3,5-dihydroxyheptanoic acid of formula I

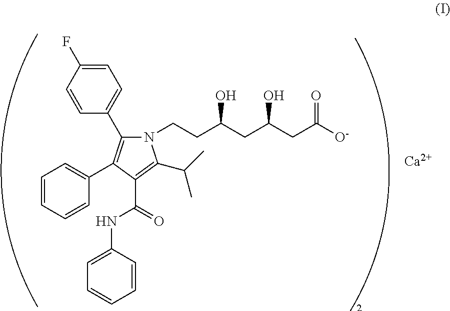

known under the non-proprietary name atorvastatin (I), possibly also the calcium salt of atorvastatin in the text below, is produced in accordance with published patents (U.S. Pat. Nos. 4,681,893 and 5,273,995). This medicament is an important representative of hypo-lipidemic and hypo-cholesteric medicaments.

Atorvastatin belongs to the group of anti-hyperlipidemically effective substances described in the patent specification No. EP 247633.

In particular, the calcium salt of atorvastatin and its specific method of preparation were described in the patent specification No. EP 409281.

A number of other patent applications have dealt with the preparation method of atorvastatin in its various crystalline or amorphous forms.

Patent applications WO 9703958 and WO 9703959, describing stable crystalline forms of the calcium salt, and WO 9703960 A, describing the process of their transformation to an amorphous substance, are worth mentioning.

The last mentioned application shows that the amorphous product is desirable from the point of view of the speed of releasing of the active substance from the pharmaceutical composition. It may be different in the case of crystals, which would bring uncertainty into the final effect of the atorvastatin-containing product.

Atorvastatin, including its salts, mainly its calcium salt, which is used in commercial products, has turned out to be a substance with problematic stability. In the case of using the amorphous form of atorvastatin the problem of stability is even more urgent (WO 9703958).

Patent application No. WO 9416693 describes several influences that may have a negative impact on stability of a pharmaceutical composition of atorvastatin, namely heat, acidic environment, humidity and light.

In accordance with the above mentioned application, stabilization of the pharmaceutical composition is achieved by addition of basic salts of Ca, Mg or Li ions. This way the above mentioned negative influences are eliminated. However, the stability values of the pharmaceutical composition mentioned in this application approximate the limit of acceptability and do not comply with increasing requirements for the quality of pharmaceutical products.

However, application No. WO 0035425 shows that the use of a too basic reagent may cause local irritation of mucous membranes in the digestive tract, which may lead to indigestion in patients. This is why the application recommends replacing the previously known salts with the finer buffers. The stability of the products is allegedly good and a minimum quantity of degradation products has been found. The application does not contain particular data of their quantity and methods of their measurement.

However, application No. WO 02072073 shows that too low pH impairs releasing of the active substance. Especially, if one considers the acidic environment of the stomach, the buffers suggested in the previous application would have to be present in a very high concentration. The composition in accordance with the quoted application should, after dissolution in simulated gastric juice, increase pH to pKa+1 of atorvastatin acid. This can be achieved either with a large quantity of the buffer requiring a very large tablet or by replacing the buffer with a stronger base, e.g. MgO, which however involves the possibility of local mucous membrane irritation.

The described invention deals with such a composition that reduces the possibility of mucous membrane irritation, achieves excellent values of releasing the effective substance, is satisfactory with regard to stability and has acceptable other physical parameters in addition.

### DISCLOSURE OF INVENTION

The invention has, as its object, a pharmaceutical composition containing the active substance atorvastatin in the form of an oblong-shaped tablet with the length of 5 to 22 mm and the width of 2 to 11 mm or a round tablet with the diameter of 3 to 16 mm, the core of which is constituted by compressed granulate and contains:
i. Atorvastatin and/or at least one physiologically acceptable salt thereof in the quantity of 5 to 10% by weight, related to pure atorvastatin;
ii. An organic or inorganic base selected from meglumine or an alkali metal hydroxide or their combination in the quantity of 0.01 to 7% by weight;
iii. A pharmaceutically acceptable filler in the quantity of 20 to 90% by weight;
iv. A disintegrant in the quantity of 0.5 to 50% by weight; provided with a coat which makes up 1 to 15% of the weight of the core, the selected base being uniformly distributed in the tablet core by means of spraying the same on the solid mixture in the granule production process.

Another object is represented by a process of manufacturing such composition, consisting in carrying out the following steps:
i. Mixing a mixture of atorvastatin, a filler and a disintegrant;
ii. Dissolving the base in a mixture of water and a C₁ to C₃ alcohol in the quantity of 10:90 to 90:10 (water/alcohol, by weight);
iii. Spraying the dry mixture with the base solution either in a masticating or fluidizing device;
iv. Adapting the size of particles of the resulting granulate, best by re-sieving to the granule size of 0.1 to 1.5 mm;
v. Adding other extragranular components to the granulate that will further improve its capability to be compressed into tablets;
vi. Compressing the mixture; and vii. Applying a coat on the compressed tablets.

The invention also provides a composition produced by means of this procedure, which represents an advantageous solution of the subject matter of the invention.

### DETAILED DESCRIPTION

Experiments with many various bases, used as stabilizing agents, have shown that a preferable base should get very quickly dissolved in the gastric fluid, which causes homogenization and eliminates its local concentrating and subsequent irritation of the mucous membrane. This has been achieved by uniform distribution of the base in the core of the tablet of the invention. This can be obtained by spraying the filler with a solution of the given base.

Out of the large number of bases that have been tested in this manner, meglumine or sodium hydroxide have turned out to be suitable. Although in the case of the hydroxide it would be the case of a strong base, just a low concentration could be used and with uniform distribution and ensuring of a quick decomposition of the tablet the possibility of a contact of NaOH with the stomach wall was eliminated.

For the realization of the invention the oblong shape of the tablet with the length of 5 to 22 mm and the width of 2 to 11 mm has proved to be advantageous. Benefits of the oblong shape are significant especially for higher doses of atorvastatin. However, the invention can also be realized in round tablets with the diameter of 3 to 16 mm.

It has also proved to be advantageous in the case of atorvastatin if the filler was mixed also with the active substance, the binder and at least a part of the disintegrant used and this mixture was sprayed with a solution of a base in a masticating or fluidizing device, thus generating a granulate that could be used for further production of tablets. The size of granules advantageously varies between 0.1 and 1.5 mm. However, for successful production of tablets the granulate must also contain dust fractions with the particle size lower than 0.1 mm.

Preferably, the proportion of granules with the above mentioned size is 50 to 80% of the total granulate weight and 20 to 50% by weight of the dust particles. Individual components of the table are either located inside the granules or in the space between the granules.

By compressing the granulate and coating the same a coated tablet is produced whose properties may also be characterized by the tablet hardness of 50 to 300 N, disintegration in 10 to 600 s and the releasing rate at pH 4.5 at 75 rpm under conditions defined by Ph. Eur., wherein more than 60% of the active substance are released in 30 minutes.

The quantity of the base used varied between 0.01 and 7% by weight. Preferably, the base quantity was selected in the range between 0.1 and 5%, the quantity of NaOH being below 1%, while meglumine is in the range of 0.5 to 5%. For example, 0.25% of NaOH and 1% of meglumine proved to be suitable.

As the previous publications concluded, the release rate of the active substance—atorvastatin depends on its form present in the tablet. Experiments with various crystalline forms of atorvastatin showed in our case as well that it was not possible to achieve such a release rate as with amorphous atorvastatin. Hence the composition in accordance with the invention advantageously contains amorphous atorvastatin, especially preferably its calcium salt.

Stabilization of amorphous atorvastatin, as expected, has turned out to be more problematic than in the case of the crystalline form. Although both the bases ensure good stabilization, the product proved to be more stable if packed in an atmosphere with a lower partial pressure of oxygen. The partial pressure should be lower than at least 20 kPa. Significantly better results are achieved by further reductions of the partial pressure below 5, beneficially 2, preferably 1 kPa. If the pharmaceutical composition is packed in an atmosphere with the partial pressure of oxygen of 0.4 kPa or lower, no oxidation products appear at all.

The above mentioned need of quick decomposition of the tablet is related to the quantity and quality of the disintegrant used. For the realization of the invention it is necessary to use the disintegrant in a quantity of 0.5 to 50%, advantageously 3 to 25% (weight percentage).

Further, using a combination of two types of disintegrants proved to be advantageous. The first one can be selected from the group of classical disintegrants, e.g. starch, pre-gelatinized starch, alginate, microcrystalline cellulose or low-substituted hydroxypropylcellulose. The other group contains so-called super disintegrants selected from the group of crosscarmellose, crosspovidone or the sodium salt of carboxymethyl starch.

Super disintegrants for the composition are selected in the quantity of 0.5 to 2.5% and the classical disintegrants in the quantity of 5 to 25% (weight percentage).

In a preferable embodiment crosscarmellose or crosspovidone is selected as the super disintegrant and low-substituted hydroxypropylcellulose as the classical disintegrant, which at the same time serves as the binder for the production of granulate. In a preferable embodiment the LH21 type of hydroxypropylcellulose was used.

The selection of the location of the disintegrant is also important for the effect of the subject matter of the invention. Envisaged is locating inside the granule or in the tablet in the space between individual granules. While in the former case it ensures disintegration of the granules themselves and releasing of the active substance, in the latter case it is disintegration of the tablet into individual granules. It is clear that both the processes participate in successful releasing of the effective substance.

It has been shown that for the case of the subject matter of the invention at least a part of the disintegrant used must be inside the granules. However, it was advantageous if all the disintegrant, i.e. both the types of the disintegrants used mentioned above were located inside the granules.

In addition to the total percentage of the base contained in the tablet, the weight proportion of this base to atorvastatin has turned out to be important, which varies between 1:100 and 1:2. In this case the selected proportion obviously depends on the selection of the particular base. The proportion of meglumine to atorvastatin is selected in the range of 1:10 to 1:2, while in the case of sodium hydroxide the proportion is 1:100 to 10:100.

The principle of the invention is better elucidated in the following examples:

### Example 1

### Comparative

Coated tablets containing 10 mg of atorvastatin in the form of amorphous calcium salt with the composition specified in Table 1.

TABLE 1

| | Sample 010205 |
|---|---|
| | Quantity (mg) |
| Calcium salt of | 10.0 |
| atorvastatin | |
| Lactose monohydrate | 40.3 |
| Microcrystalline | 70.0 |
| cellulose | |
| Sodium salt of | 4.5 |
| crosscarmellose | |
| Hydroxypropylcellulose | 14.0 |
| Silicon dioxide | 0.5 |
| Magnesium stearate | 0.7 |

From the calcium salt of atorvastatin, lactose, microcrystalline cellulose and hydroxypropylcellulose a granulate was prepared by wet granulation, to which the sodium salt of crosscarmelose, silicon dioxide and magnesium stearate were added in an extra-granular manner after drying and sieving. This matter was compressed into tablets having the weight of 140 mg. The tablets were subsequently coated with standard varnish based on hydroxypropylmethylcellulose. The coated tablets were adjusted in Al/Al blister packing in a nitrogen atmosphere. The tablets were stored at the temperature of 40° C. and the relative humidity of 75% for 6 months.

### Results of Stability Tests

| Test - impurities, | | | |
|---|---|---|---|
| HPLC (%) | 0 months | 3 months | 6 months |
| Lactone | 0.11 | — | 1.07 |
| RTT 1.32 | 0.12 | — | 0.20 |
| Sum | 0.77 | — | 1.63 |

### Example 2

Optimization of the formulation—stabilization of the dosage form using meglumine as the basic substance.

Coated tables with the content of 10 mg of atorvastatin in the form of amorphous calcium salt with the composition specified in Table 2.

TABLE 2

| | Sample 020106 | Sample 030106 | Sample 060406 |
|---|---|---|---|
| | Quantity (mg) | Quantity (mg) | Quantity (mg) |
| Calcium salt of | 10.0 | 10.0 | 10.0 |
| atorvastatin | | | |
| Lactose monohydrate | 38.9 | 36.1 | 37.5 |
| Microcrystalline | 70.0 | 70.0 | 70.0 |
| cellulose | | | |
| Sodium salt of | 4.5 | 4.5 | 4.5 |
| crosscarmellose | | | |
| Hydroxypropyl- | 14.0 | 14.0 | 14.0 |
| cellulose | | | |
| Meglumine | 1.4 | 4.2 | 2.8 |
| Silicon dioxide | 0.5 | 0.5 | 0.5 |
| Magnesium stearate | 0.7 | 0.7 | 0.7 |

The calcium salt of atorvastatin was mixed with lactose, microcrystalline cellulose, hydroxypropylcellulose and a part of the sodium salt of crosscarmellose in a masticating device and was granulated with an alcoholic-aqueous solution of meglumine. The produced granulate was mixed with the sodium salt of crosscarmellose, silicon dioxide and magnesium stearate after drying and sieving. This matter was compressed into tablets with the weight of 140 mg. The tablets were subsequently coated with standard varnish based on hydroxypropylmethylcellulose. The coated tablets were adjusted in Al/Al blister packing in a nitrogen atmosphere. The tablets were stored at the temperature of 40° C. and the relative humidity of 75% for 6 months.

Results of stability tests—sample 020106

| Test - impurities, | | | |
|---|---|---|---|
| HPLC (%) | 0 months | 3 months | 6 months |
| Lactone | 0.07 | 0.18 | 0.25 |
| RTT 1.32 | 0.06 | 0.06 | 0.06 |
| Sum | 0.55 | 0.61 | 0.67 |

Results of stability tests—sample 030106

| Test - impurities, | | | |
|---|---|---|---|
| HPLC (%) | 0 months | 3 months | 6 months |
| Lactone | <0.05 | 0.07 | 0.12 |
| RTT 1.32 | 0.06 | 0.05 | 0.06 |
| Sum | 0.51 | 0.51 | 0.58 |

Results of stability tests—sample 060406

| Test - impurities, | | | |
|---|---|---|---|
| HPLC (%) | 0 months | 3 months | 6 months |
| Lactone | 0.05 | 0.05 | 0.15 |
| RTT 1.32 | 0.07 | 0.08 | 0.07 |
| Sum | 0.50 | 0.56 | 0.64 |

### Example 3

Optimization of the formulation—stabilization of the dosage form using sodium hydroxide as the basic substance.

Coated tables with the content of 10 mg of atorvastatin in the form of amorphous calcium salt with the composition specified in Table 3.

TABLE 3

| | Sample 040106 |
|---|---|
| | Quantity (mg) |
| Calcium salt of | 10.0 |
| atorvastatin | |
| Lactose monohydrate | 39.9 |
| Microcrystalline | 70.0 |
| cellulose | |
| Sodium salt of | 4.5 |
| crosscarmellose | |
| Hydroxypropylcellulose | 14.0 |
| Sodium hydroxide | 0.4 |
| Silicon dioxide | 0.5 |
| Magnesium stearate | 0.7 |

The calcium salt of atorvastatin was mixed with lactose, microcrystalline cellulose, hydroxypropylcellulose and a part of the sodium salt of crosscarmellose in a masticating device and was granulated with an alcoholic-aqueous solution of sodium hydroxide. The produced granulate was mixed with the sodium salt of crosscarmellose, silicon dioxide and magnesium stearate after drying and sieving. This matter was compressed into tablets with the weight of 140 mg. The tablets were subsequently coated with standard varnish based on hydroxypropylmethylcellulose. The coated tablets were adjusted in Al/Al blister packing in a nitrogen atmosphere. The tablets were stored at the temperature of 40° C. and the relative humidity of 75% for 6 months.

Results of stability tests—sample 040106

| Test - impurities, | | | |
|---|---|---|---|
| HPLC (%) | 0 months | 3 months | 6 months |
| Lactone | 0.05 | 0.18 | 0.33 |
| RTT 1.32 | 0.06 | 0.05 | 0.09 |
| Sum | 0.46 | 0.63 | 0.91 |

### Example 4

Optimization of the formulation—acceleration of release of the active substance from the dosage form at pH 4.5.

Coated tables with the content of 40 mg of atorvastatin in the form of amorphous calcium salt with the composition specified in Table 4

TABLE 4

| | | Sample V/ | Sample V/ |
|---|---|---|---|
| | Sample 071106 | 011106 | 090107 |
| | Quantity (mg) | Quantity (mg) | Quantity (mg) |
| Calcium salt of | 40.0 | 40.0 | 40.0 |
| atorvastatin | | | |
| Lactose monohydrate | 155.6 | 155.6 | 127.6 |
| Microcrystalline | 280.0 | 280.0 | 280.0 |
| cellulose | | | |
| Sodium salt of | 6.0 + 12.0 | 28.0 + 0 | 56.0 + 0 |
| crosscarmellose | | | |
| hydroxypropyl- | 56.0 | 56.0 | 56.0 |
| cellulose | | | |
| Meglumine | 5.6 | 5.6 | 5.6 |
| Silicon dioxide | 2.0 | 2.0 | 2.0 |
| Magnesium stearate | 2.8 | 2.8 | 2.8 |

The calcium salt of atorvastatin was mixed with lactose, microcrystalline cellulose, hydroxypropylcellulose and a part of the sodium salt of crosscarmellose in a masticating device and was granulated with an alcoholic-aqueous solution of sodium hydroxide. The produced granulate was mixed with the sodium salt of crosscarmellose, silicon dioxide and magnesium stearate after drying and sieving. This matter was compressed into tablets with the weight of 560 mg.

The tablets produced this way were subject to a dissolution test at pH 4.5 and 75 rpm.

The results of releasing of the active substance from the tablets (%) are specified in the following Table:

| | 10 min | 20 min | 30 min | 45 min |
|---|---|---|---|---|
| Sample 071106 | 54.3 | 59.3 | 62.7 | 65.5 |
| Sample V/011106 | 74.1 | 80.6 | 82.7 | 84.8 |
| Sample V/090107 | 80.5 | 85.5 | 87.0 | 87.9 |

1. A pharmaceutical composition comprising atorvastatin, in the form of oblong-shaped tablets with length of 5 to 22 mm and width of 2 to 11 mm or round tablets with the diameter of 3 to 16 mm, having a core which is constituted of compressed granulate and contains:
i. Atorvastatin and/or at least one physiologically acceptable salt thereof in the quantity of 5 to 10% by weight, related to pure atorvastatin;
ii. An organic or inorganic base selected from meglumine or an alkali metal hydroxide or a combination thereof, in the quantity of 0.01 to 7% by weight;
iii. A pharmaceutically acceptable filler in the quantity of 20 to 90% by weight; and
iv. A disintegrant in the quantity of 0.5 to 50% by weight; and a coat that makes up 1 to 15% of the weight of the core, wherein said base being uniformly distributed in the tablet core by means of spraying the same on the solid mixture in the granule production process.

2. The composition in accordance with claim 1, wherein the pharmaceutical composition it is a tablet having an oblong shape.

3. The pharmaceutical composition in accordance with claim 1, wherein said pharmaceutical composition it contains atorvastatin and/or its salt in an amorphous form.

4. The pharmaceutical composition in accordance with claim 1, wherein 50 to 80% by weight of the granulate consist of granules, their size varies in the range of 0.1 to 1.5 mm, and 20 to 50% of the granulate are dust fractions smaller than 0.1 mm.

5. The pharmaceutical composition in accordance with claim 1, wherein its hardness value is 50 to 300 N, disintegration value is 10 to 600 s and more than 60% of the active substance are released in 30 minutes at pH 4.5 at 75 rpm under conditions defined by Ph. Eur.

6. The pharmaceutical composition in accordance with claim 1, wherein it is it packed and kept in blister packing or a vial.

7. The pharmaceutical composition in accordance with claim 6, wherein a partial pressure of oxygen P_{O} lower than 20 kPa is maintained in the respective package.

8. The pharmaceutical composition in accordance with claim 7, wherein a partial pressure of oxygen P_{O} lower than 5 kPa is maintained in the respective package.

9. The pharmaceutical composition in accordance with claim 8, wherein a partial pressure of oxygen P_{O} lower than 2 kPa is maintained in the respective package.

10. The pharmaceutical composition in accordance with claim 9, wherein a partial pressure of oxygen P_{O} lower than 1 kPa is maintained in the respective package.

11. The pharmaceutical composition in accordance with claim 10, wherein a partial pressure of oxygen P_{O} lower than 0.4 kPa is maintained in the respective package.

12. The pharmaceutical composition in accordance with claim 1, wherein a part of the disintegrant used is located inside the granules compressed into the tablet and the second part is located in the space between granules in the tablet.

13. The pharmaceutical composition in accordance with claim 1, wherein that all the disintegrant is located inside the granules compressed into tablets.

14. The pharmaceutical composition in accordance with claim 1, wherein a combination of two types of disintegrants is used, i.e. classical disintegrants from the group of starches, pre-gelatinized starches, alginates, microcrystalline cellulose or low-substituted hydroxypropylcellulose and a so-called super disintegrants from the group of crosscarmellose, crosspovidone or the sodium salt of carboxymethyl starch.

15. The pharmaceutical composition in accordance with claim 14, wherein it is a combination of the disintegrants crosscarmellose or crosspovidone and low-substituted hydroxypropylcellulose.

16. The pharmaceutical composition in accordance with claim 15, wherein crosscarmellose or crosspovidone is present in the quantity of 0.5 to 25% by weight and low-substituted hydroxypropylcellulose is present in the quantity of 5 to 25% by weight.

17. The pharmaceutical composition in accordance with claim 16, wherein crosscarmellose or crosspovidone is present in the quantity of 1.5 to 10% by weight and low-substituted hydroxypropylcellulose is present in the quantity of 7 to 15% by weight.

18. The pharmaceutical composition in accordance with claim 1, wherein meglumine is in a proportion to atorvastatin of 1:2 to 1:10.

19. The pharmaceutical composition in accordance with claim 18, wherein it has been obtained by a procedure comprising:
i. Mixing a mixture of atorvastatin, a filler, a disintegrant and a binder;
ii. Dissolving meglumine in a mixture of water and a C₁ to C₃ alcohol in a weight ratio of 10:90 to 90:10;
iii. Spraying the dry mixture with the solution of meglumine in a masticating or fluidizing device;
iv. Adapting the size of particles of the resulting granulate, by re-sieving to the granule size of 0.1 to 1.5 mm;
v. Adding other extragranular components to the granulate;
vi. Compressing the mixture;
vii. Applying a coat on the compressed tablets.

20. The pharmaceutical composition in accordance with claim 1, wherein it contains sodium hydroxide in the proportion to atorvastatin of 1:100 to 10:100.

21. The pharmaceutical composition in accordance with claim 20, wherein it has been obtained by a procedure comprising:
i. Mixing a mixture of atorvastatin, a filler, a disintegrant and a binder;
ii. Dissolving sodium hydroxide in a mixture of water and a C₁ to C₃ alcohol in a weight ratio of 10:90 to 90:10;
iii. Spraying the dry mixture with the solution of sodium hydroxide in a masticating or fluidizing device;
iv. Adapting the size of particles of the resulting granulate, preferably by re-sieving to the granule size of 0.1 to 1.5 mm;
v. Adding other extragranular components to the granulate;
vi. Compressing the mixture;
vii. Applying a coat on the compressed tablets.

22. The pharmaceutical composition in accordance with claim 1, wherein the film-forming substance hydroxypropylmethylcellulose, hydroxypropylcellulose, acrylate-based substances or their mixtures are used in the coat.
